(19) United States
(12) Patent Application Publication
(10) Pub. No.: US 2007/0077222 A1
(43) Pub. Date: Apr. 5, 2007

(54) Composition and treatment for hair loss

(76) Inventors: Steven D. Shapiro, Tequesta, FL (US); Michael Ted Borenstein, Jupiter, FL (US); Joseph T. Owens, Juno Beach, FL (US); Bert P. Gould, New York, NY (US)

Correspondence Address: MARK YOUNG, P.A., 12086 FORT CAROLINE ROAD, UNIT 202, JACKSONVILLE, FL, 32225, US

(21) Appl. No.: 11/242,207

(22) Filed: Oct. 3, 2005

(51) Int. Cl. A61K 8/97; A61K 36/889

(52) U.S. Cl. 424/74; 424/727

(57) ABSTRACT

A topical hair treatment or a topical dermatological or cosmetic composition includes a liquid having approximately 0.2 to 0.5 mg/mL of a xanthene derivative. The xanthene derivative can be a methylxanthene. The methylxanthene can be theophylline, caffeine, and/or theobromine. Preferably, the methylxanthene is caffeine selected from hydrous caffeine, caffeine salts, and complexes dissociated to yield caffeine. Caffeine is present in an amount of approximately 0.3 mg/mL. There is also provided a saw palmetto berry derivative present in the liquid in an amount of approximately 0.02 to 0.05 mg/mL. Preferably, the saw palmetto berry derivative is saw palmetto berry extract, in particular, hydrous saw palmetto berry extract. The saw palmetto berry extract is present in said liquid in an amount of approximately 0.03 mg/mL. The liquid can be a shampoo, a conditioner, and/or a combination shampoo and conditioner.

### STATEMENT REGARDING FEDERALLY SPONSORED RESEARCH OR DEVELOPMENT

n/a

### FIELD OF THE INVENTION

The present invention lies in the field of medical treatments and preparations. In particular, the present invention is directed to a topical treatment for human hair loss.

### BACKGROUND OF THE INVENTION

Hair loss is affecting more people each year based, primarily, upon the aging of the population and the rise of the baby-boomers. The loss is not restricted to men. Women are affected as well. The loss is not just physical, but is extremely emotional. Accordingly, the hair loss prevention or hair growth industry has, in the past decade, become an approximately 80 billion dollar market worldwide.

Male pattern hair loss begins typically between the ages of 30 to 50 years old. However, when the loss is hereditary, it may begin earlier. In some families, it begins during the twenties and even the teens. One predominant mechanism of male pattern hair loss is due to the effects of dihydrotestosterone (DHT) on the hair follicle. When DHT is present, its adverse affect leads to progressive hair miniaturization and, ultimately, to loss of the hair follicle.

Oral and topical medications have been used to treat this emotionally distressing condition. Topical Minoxidil (ROGAINE®) and oral Finasteride (PROPECIA®) are FDA approved prescription medications for male pattern hair loss, referred to in the art as “Androgenic” alopecia. The mechanism of action for Minoxidil is not known. Nonetheless, Minoxidil in a 2% concentration became a non-prescription product sold over-the-counter for treatment of hair loss.

Topical Minoxidil has risks of localized allergy and irritation and may lead to systemic absorption with lowered blood pressure and its associated symptoms of headache, fatigue, and dizziness.

Oral Finasteride is a 5-alpha-reductase inhibitor that decreases levels of DHT. This medication has risks associated therewith, including, for example, decreased libido, decreased volume of the male's ejaculate, and affects on the level of the Prostate Specific Antigen, which may affect screening for prostate cancer. Both Minoxidil and Finasteride have other side effects and unpredictable clinical-responses. Therefore, the-search for a better, safer product is ongoing.

Topical medications have a safer profile than oral medications because the latter have a higher incidence of systemic side effects. However, to date, topical medications, including Minoxidil, have been used with mixed results, at best. In theory, topical medications targeted at testosterone derivatives should block the effects of dihydrotestosterone on its receptor that triggers male pattern hair loss but, to date, none have been available to the public.

It would be desirable, therefore, to provide a topical preparation that treats hair loss and stimulates hair growth.

### SUMMARY OF THE INVENTION

The present invention provides a novel and non-obvious method and system for treating hair loss.

Both SPBE and caffeine have been found to block the effects of DHT. Saw palmetto berry extract is a potent naturally occurring DHT blocker that can be used to lower DHT and, thereby, treat hair loss. Saw palmetto berry extract can be used in combination with a low concentration of an alkaloid, specifically, a xanthene derivative or one of the family of methylxanthenes. While theophylline, caffeine, and theobromine are three closely related alkaloids that are methyxanthenes, in particular, caffeine can be used to further lower DHT and, thereby, treat hair loss. In individuals with high caffeine intake, lower testosterone levels were found suggesting that caffeine reduces levels of testosterone, a precursor of DHT. Bishop D T, Meikle A W, Slattery M L, Stringham J D, Ford M H, West D W. The effect of nutritional factors on sex hormone levels in male twins. Genet Epidemiol 1988; 5(1):43-59. Caffeine can be used separately or together with saw palmetto berry extract.

The invention of the instant application takes advantage of the natural properties of xanthene derivatives and derivatives of saw palmetto berry and, taken alone or in combination, provides a hair loss product that does not need a prescription for distribution and use. The formulation achieves synergistic results if the saw palmetto berry is an extract applied in a topical preparation and the xanthene, methylxanthene, or caffeine is also applied in the form of a topical preparation.

Even though saw palmetto berry derivatives, xanthene, and methylxanthenes, in particular, caffeine, are naturally occurring and well-known, they have not been known as hair loss prevention components or used for the purpose of hair growth. The invention of the instant application uses the xanthene or a combination of the xanthene and palmetto berry derivatives in a topical product. These two components have been proven to have minimal side effects when used internally. Therefore, the topical preparation of the present invention should be safer than current preparations because the active ingredient is localized (versus systemic). As topical products, saw palmetto berry derivatives, in particular, hydrous saw palmetto berry extract, and xanthene, in particular, hydrous caffeine, are targeted directly towards a localized problem (to the area where hair loss is occurring) and are expected to reduce the effects of DHT to slow, stop, or eliminate the male pattern hair loss process.

With the foregoing and other objects in view, there is provided, in accordance with the invention, a topical hair treatment, including a liquid being one of a shampoo, a conditioner, and a combination shampoo and conditioner, said liquid having approximately 0.2 to 0.5 mg/mL of a xanthene derivative.

In accordance with another feature of the invention, the xanthene derivative is a methylxanthene. The methylxanthene can be theophylline, caffeine, and theobromine. Preferably, the methylxanthene is caffeine selected from hydrous caffeine, caffeine salts, and complexes dissociated to yield caffeine.

In accordance with a further feature of the invention, the caffeine is present in said liquid in an amount of approximately 0.3 mg/mL.

In accordance with an added feature of the invention, there is also provided a saw palmetto berry derivative present in said liquid in an amount of approximately 0.02 to 0.05 mg/mL. Preferably, the saw palmetto berry derivative is saw palmetto berry extract, in particular, hydrous saw palmetto berry extract.

In accordance with an additional feature of the invention, the saw palmetto berry extract is present in said liquid in an amount of approximately 0.03 mg/mL.

With the objects of the invention in view, there is also provided a topical hair treatment, including a liquid being one of a shampoo, a conditioner, and a combination shampoo and conditioner, said liquid having approximately 0.2 to 0.5 mg/mL of caffeine and approximately 0.02 to 0.05 mg/mL of saw palmetto berry extract.

With the objects of the invention in view, there is also provided a topical hair treatment, including a liquid comprising approximately 0.2 to 0.5 mg/mL of caffeine. The liquid can be a shampoo, a conditioner, or a combination shampoo and conditioner.

With the objects of the invention in view, there is also provided a topical hair treatment, including a liquid including approximately 0.2 to 0.5 mg/mL of caffeine and approximately 0.02 to 0.05 mg/mL of a saw palmetto berry derivative. The liquid can be a shampoo, a conditioner, and/or a combination shampoo and conditioner.

With the objects of the invention in view, there is also provided a topical dermatological or cosmetic composition, including a liquid comprising approximately 0.2 to 0.5 mg/mL of caffeine. The liquid can be a shampoo, a conditioner, and/or a combination shampoo and conditioner.

With the objects of the invention in view, there is also provided a topical dermatological or cosmetic composition, comprising a liquid comprising approximately 0.2 to 0.5 mg/mL of caffeine and approximately 0.02 to 0.05 mg/mL of a saw palmetto berry derivative. The liquid can be a shampoo, a conditioner, and/or a combination shampoo and conditioner.

A relationship between male testosterone levels and hair loss has already been well-described in the pathogenesis of this emotionally distressing condition. Blockers of testosterone and its metabolite, dihydrotestosterone (DHT), have been used systemically to help male pattern hair loss (e.g., Finasteride). Saw palmetto berry extract has been described with respect to its Finasteride-type effects on DHT in Lowe F C, Fagelman E. Phytotherapy in the treatment of benign prostatic hyperplasia. Current Opin Urol 2002;12:15-18, and in Prager N, Bickett, K, French N, Marcovici G. A randomized, double-blind, placebo-controlled trial to determine the effectiveness of botanically derived inhibitors of 5-alpha reductase in the treatment of androgenetic alopecia. Alt Comp Med 2002; 8(2):148-152. However, to date, it has not been suggested or used in a topical preparation. In the formulation where the present invention is in a topical preparation, it is expected that the formulation blocks DHT's activity in the male pattern hair loss process.

Caffeine intake has been shown in Bishop D T, Meikle A W, Slattery M L, Stringham J D, Ford M H, West D W. The effect of nutritional factors on sex hormone levels in male twins. Genet Epidemiol 1988; 5(1):43-59, to reduce DHT. In addition, topical caffeine has been shown in Van de Sandt J J, van Burgsteden J A, Cage S, Carmichael P L, Dick I, Kenyon S, Korinth G, Larese F, Limasset J C, Maas W J, Montomoli L, Nielsen J B, Payan J P, Robinson E, Sartorelli P, Schaller K H, Wilkinson S C, Williams F M. In vitro predictions of skin absorption of caffeine, testosterone, and benzoic acid: a multi-centre comparison study, and Tanada N, Kashimura S, Kageura M, Hara K. Utility of caffeine analysis for forensic hair discrimination. Nippon Hoigaku zasshi 1998; 52 (4):233-7, to penetrate the skin and hair. But, topical preparations using caffeine in high concentrations of 1% of weight per unit volume or more has a vasoconstrictive effect. Vasoconstriction is understood to decrease blood flow to a hair follicle and, thereby, inhibit hair growth. Thus, using caffeine for hair loss prevention is not suggested in the art. Nonetheless, the present invention uses a concentration of caffeine that avoids vasoconstriction, but maintains its reducing effects on DHT.

As used herein, a dermatological composition is any product that is used in association with the integumentary system, i.e., skin, hair, or nails. As used herein, a cosmaceutical composition is a product that is available to the public over-the-counter or without a prescription to improve the integumentary system.

Topical products have the advantage of potentially reducing unwanted systemic side effects and working locally at the source of the hair loss problem. The present invention uses one or two products, in particular, saw palmetto berry extract and caffeine, in order to reduce DHT and, thereby, treat hair loss. It is possible that the reduction of DHT using the formulation of the present invention could, potentially, grow hair. The process is better than previous treatments because, in preferred formulations, it contains products that, independently, each is expected to block the effects of DHT.

In a preferred embodiment of the present invention, the formulation for treatment of hair loss is a topical preparation.

The topical caffeine formulation of the present invention can be used by both men and women. There are a number of medications that present possible detriment to women who are pregnant. The possibility of such detriment is increased when caffeine is combined with saw palmetto berry extract. Thus, precautions should be taken by women using the formulations and methods of the present invention.

Other features that are considered as characteristic for the invention are set forth in the appended claims.

Although the invention is illustrated and described herein as embodied in a composition and treatment for hair loss, it is, nevertheless, not intended to be limited to the details described because various modifications and structural changes may be made therein without departing from the spirit of the invention and within the scope and range of equivalents of the claims.

The construction and method of operation of the invention, however, together with additional objects and advantages thereof, will be best understood from the following description of specific embodiments.

### DETAILED DESCRIPTION OF THE PREFERRED EMBODIMENTS

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention.

While the specification concludes with claims defining the features of the invention that are regarded as novel, it is believed that the invention will be better understood from a consideration of the following description in conjunction with the drawing figures, in which like reference numerals are carried forward.

It is to be understood that the terminology used herein is for the purpose of describing particular embodiments only and is not intended to be limiting. It must be noted that, as used in the specification and the appended claims, the singular forms “a,” “an,” and “the” include plural references unless the context clearly dictates otherwise.

Treat or treating, as used in the specification and appended claims refers to the slowing, stopping, or eliminating of hair loss (e.g., male pattern hair loss).

Shampoo, as used in the specification and appended claims, refers to compositions that are known in the art as a shampoo. Typically, a shampoo is a composition that is applied to the scalp or hair for the general purpose of cleaning the scalp or hair. Shampoo, as used in the specification and appended claims, comprises known components commonly used in the art (e.g., soaps, cleansers, detergents, surfactants, anti-dandruff agents, etc.).

Conditioner, as used in the specification and appended claims, refers to compositions that are known in the art as a conditioner. Typically, a conditioner is a composition that is applied to the scalp or hair for the general purpose of conditioning the scalp or hair (e.g., improving texture and tone, de-tangling, lubricating, softening, moisturizing, thickening, improving shine, etc.). Conditioner, as used in the specification and appended claims, comprises known components commonly used in the art (e.g., silicone compounds, polyalkylene glycols, etc.). Known conditioners are often either the leave-in type or the rinse-off type.

The combination of shampoo and conditioner, as used in the specification and appended claims, refers to compositions that combine the characteristics of a shampoo and conditioner.

One formulation of the present invention is a shampoo, conditioner, or combination thereof comprising approximately 0.2 to 0.5, 0.25 to 0.35, 0.3 to 0.5, or 0.4 to 0.5 mg/mL of a methylxanthene to a shampoo or to a conditioner. An example of this type of formulation would comprise approximately 0.3 mg/mL of caffeine.

Another formulation of the present invention is a shampoo, conditioner, or combination thereof comprising 0.2 to 0.5, 0.25 to 0.35, 0.3 to 0.5, or 0.4 to 0.5 mg/mL of a methylxanthene and 0.02 to 0.05, 0.025 to 0.035, 0.03 to 0.05, 0.04 to 0.05 mg/mL saw palmetto berry extract. An example of this type of formulation would comprise approximately 0.03 mg/mL saw palmetto berry extract and approximately 0.3 mg/mL caffeine.

The instant invention also includes a method of treatment for hair loss comprising wetting the scalp with water, applying the shampoo (or combination shampoo/conditioner) to the scalp, massaging it into the skin of the scalp (e.g., using a circular motion), and, then, rinsing the shampoo out of the scalp. The treatment can be performed on a daily basis, though other treatment timings could be used (e.g., every other day, weekly, etc.). The present invention also provides a method of treatment for hair loss comprising applying the conditioner (e.g., a leave-in conditioner) to the scalp (e.g., by using the hands). The conditioner can be combed or brushed in throughout the desired areas of the scalp. When using a conditioner of the present invention, it can be left in (e.g., a leave-in conditioner) or rinsed off (e.g., a rinse-off conditioner).

The foregoing description and accompanying drawings illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

We claim:

1. A topical hair treatment, comprising:
a liquid being one of a shampoo, a conditioner, and a combination shampoo and conditioner, wherein a xanthene derivative is present in said liquid in an amount of approximately 0.2 to 0.5 mg/mL.

2. The topical hair treatment according to claim 1, wherein the xanthene derivative is a methylxanthene.

3. The topical hair treatment according to claim 2, wherein the methylxanthene is selected from the group consisting of theophylline, caffeine, and theobromine.

4. The topical hair treatment according to claim 2, wherein the methylxanthene is caffeine.

5. The topical hair treatment according to claim 4, wherein the caffeine is selected from the group consisting of hydrous caffeine, caffeine salts, and complexes dissociated to yield caffeine.

6. The topical hair treatment according to claim 4, wherein said caffeine is present in said liquid in an amount of approximately 0.3 mg/mL.

7. The topical hair treatment according to claim 1, further comprising a saw palmetto berry derivative present in said liquid in an amount of approximately 0.02 to 0.05 mg/mL.

8. The topical hair treatment according to claim 7, wherein said saw palmetto berry derivative is saw palmetto berry extract.

9. The topical hair treatment according to claim 8, wherein said saw palmetto berry extract is hydrous saw palmetto berry extract.

10. The topical hair treatment liquid according to claim 8, wherein said saw palmetto berry extract is present in said liquid in an amount of approximately 0.03 mg/mL.

11. A topical hair treatment, comprising:
a liquid being one of a shampoo, a conditioner, and a combination shampoo and conditioner, said liquid comprising:
approximately 0.2 to 0.5 mg/mL of caffeine; and
approximately 0.02 to 0.05 mg/mL of saw palmetto berry extract.

12. The topical hair treatment according to claim 11, wherein:
said caffeine is selected from the group consisting of hydrous caffeine, caffeine salts, and complexes dissociated to yield caffeine; and
said saw palmetto berry extract is hydrous saw palmetto berry extract.

13. The topical hair treatment according to claim 11, wherein said caffeine is present in said liquid in an amount of approximately 0.3 mg/mL.

14. The topical hair treatment according to claim 11, wherein said saw palmetto berry extract is present in said liquid in an amount of approximately 0.03 mg/mL.

15. A topical hair treatment, comprising:
a liquid comprising approximately 0.2 to 0.5 mg/mL of caffeine.

16. The topical hair treatment according to claim 15, further comprising a saw palmetto berry derivative present in said liquid in an amount of approximately 0.02 to 0.05 mg/mL.

17. The topical hair treatment according to claim 15, wherein said caffeine is present in said liquid in an amount of approximately 0.3 mg/mL.

18. The topical hair treatment according to claim 16, wherein said saw palmetto berry derivative is present in said liquid in an amount of approximately 0.03 mg/mL.

19. The topical hair treatment according to claim 18, wherein said saw palmetto berry derivative is saw palmetto berry extract.

20. The topical hair treatment according to claim 19, wherein said saw palmetto berry extract is hydrous saw palmetto berry extract.

21. The topical hair treatment according to claim 15, wherein said caffeine is selected from the group consisting of hydrous caffeine, caffeine salts, and complexes dissociated to yield caffeine.

22. The topical hair treatment according to claim 15, wherein said liquid is one of a shampoo, a conditioner, and a combination shampoo and conditioner.

23. The topical hair treatment according to claim 16, wherein said liquid is one of a shampoo, a conditioner, and a combination shampoo and conditioner.

24. A topical hair treatment, comprising:
a liquid comprising;
approximately 0.2 to 0.5 mg/mL of caffeine; and
approximately 0.02 to 0.05 mg/mL of a saw palmetto berry derivative.

25. The topical hair treatment according to claim 24, wherein said caffeine is selected from the group consisting of hydrous caffeine, caffeine salts, and complexes dissociated to yield caffeine.

26. The topical hair treatment according to claim 25, wherein said caffeine is present in said liquid in an amount of approximately 0.3 mg/mL.

27. The topical hair treatment according to claim 24, wherein said saw palmetto berry derivative is saw palmetto berry extract.

28. The topical hair treatment according to claim 24, wherein said saw palmetto berry derivative is present in said liquid in an amount of approximately 0.03 mg/mL.

29. The topical hair treatment according to claim 24, wherein said liquid is one of a shampoo, a conditioner, and a combination shampoo and conditioner.

30. A topical dermatological or cosmaceutical composition, comprising:
a liquid comprising approximately 0.2 to 0.5 mg/mL of caffeine.

31. The topical dermatological or cosmaceutical composition according to claim 30, wherein the liquid is one of a shampoo, a conditioner, and a combination shampoo and conditioner.

32. A topical dermatological or cosmaceutical composition, comprising:
a liquid comprising:
approximately 0.2 to 0.5 mg/mL of caffeine; and
approximately 0.02 to 0.05 mg/mL of a saw palmetto berry derivative.

33. The topical hair treatment liquid according to claim 32, wherein the liquid is one of a shampoo, a conditioner, and a combination shampoo and conditioner.
